(19) United States
(12) Patent Application Publication
(10) Pub. No.: US 2013/0178676 A1
(43) Pub. Date: Jul. 11, 2013

(54) METHODS FOR PRODUCING LIGHT OLEFINS

(71) Applicants: Bipin Virpal Vora, Naperville, IL (US); Michael James Cleveland, Northbrook, IL (US)

(72) Inventors: Bipin Virpal Vora, Naperville, IL (US); Michael James Cleveland, Northbrook, IL (US)

(73) Assignee: UOP LLC, Des Plaines, IL (US)

(21) Appl. No.: 13/344,475

(22) Filed: Jan. 5, 2012

(51) Int. Cl. C07C 1/24

(52) U.S. Cl. 585/324; 585/639

(57) ABSTRACT

Methods for producing light olefins are provided. An exemplary method includes providing an oxygenate compound capable of converting to light olefins in a catalytic reaction or to a clean reaction intermediate compound of the catalytic reaction and converting the oxygenate compound to the clean reaction intermediate compound in a chemical reaction. The chemical reaction produces a gas-phase product comprising the clean reaction intermediate compound, unconverted oxygenate, and a reaction byproduct. The method further includes cooling the gas-phase product to condense unconverted oxygenate and reaction byproduct while maintaining the clean reaction intermediate compound in a gas phase and separating the clean reaction intermediate compound, the unconverted oxygenate, and the reaction byproduct into a first stream including the clean reaction intermediate compound and a second stream including the unconverted oxygenate and the reaction byproduct. Still further, the method includes contacting the first stream with a catalyst configured for producing light olefins.

### TECHNICAL FIELD

The present disclosure relates generally to methods for producing light olefins, and more particularly relates to methods utilizing reaction intermediate compounds for producing light olefins.

### BACKGROUND

A major portion of the worldwide petrochemical industry is concerned with the production of light olefin materials, an important raw materials for polymerization, oligomerization, alkylation, and the like reaction processes, and their subsequent use in the production of numerous chemical and polymer products. Light olefins generally include ethylene (C₂H₄), propylene (C₃H₆), and mixtures thereof. These light olefins are essential building blocks used in modern chemical and petrochemical industries. A major source for light olefins in present day refining is the steam cracking of petroleum feeds. For various reasons including geographical, economic, political, and diminished supply considerations, the art has long sought sources other than petroleum for the large quantities of raw materials that are needed to supply the demand for these light olefin materials.

The search for alternative materials for light olefin production has led to the use of oxygenates such as alcohols and, more particularly, to the use of methanol, ethanol, and higher alcohols or their derivatives, for example. Molecular sieves such as microporous crystalline zeolite and non-zeolitic catalysts, particularly silicoaluminophosphate (SAPO) materials, are known to promote the conversion of oxygenates to hydrocarbon mixtures, particularly hydrocarbon mixtures composed largely of light olefins. More specifically, U.S. Pat. No. 4,499,327 to Kaiser has shown SAPO-34 more selective for the conversion of oxygenates to light olefins. These catalysts can be employed in a variety of catalytic reactor systems for large-scale conversion of oxygenates to olefins.

During start-up operations, some current oxygenate-to-olefin catalytic reactor designs require a vaporizer to convert the oxygenate feed into a gaseous phase and a pre-heater to bring the feed to the required reaction temperature, typically about 300° C. or higher, depending on the type of oxygenate. Furthermore, where a fluidized bed reactor design is employed, before the introduction of the vaporized and heated feed into the reactor, the catalyst in the fluidized bed reactor must be brought to the desired fluidization state and to the required start-up temperature, typically between about 300-350° C. Conventionally, natural gas, methane, nitrogen or other “clean” gasses are used for fluidization, and a start-up heater is used to bring the catalyst up to temperature. As such, the current start-up procedures that are required for existing oxygenate-to-olefin catalytic reactor designs are undesirably complicated, and require expensive inputs in terms of material and energy use.

Accordingly, it is desirable to provide light olefin production systems and methods for producing light olefins that reduce the operational complexity and costs incurred during start-up procedures. Furthermore, other desirable features and characteristics will become apparent from the subsequent detailed description and the appended claims, taken in conjunction with the accompanying drawings and the foregoing technical field and background.

### BRIEF SUMMARY

Light olefin production methods are provided herein. In an exemplary embodiment, a method includes providing an oxygenate compound capable of converting to light olefins in a catalytic reaction or to a clean reaction intermediate compound of the catalytic reaction and converting the oxygenate compound to the clean reaction intermediate compound in a chemical reaction. The chemical reaction produces a gas-phase product comprising the clean reaction intermediate compound, unconverted oxygenate, and a reaction byproduct. The method further includes cooling the gas-phase product to condense unconverted oxygenate and reaction byproduct while maintaining the clean reaction intermediate compound in a gas phase and separating the clean reaction intermediate compound, the unconverted oxygenate, and the reaction byproduct into a first stream including the clean reaction intermediate compound and a second stream including the unconverted oxygenate and the reaction byproduct. Still further, the method includes contacting the first stream with a catalyst configured for producing light olefins.

In accordance with another exemplary embodiment, a method includes providing an oxygenate compound capable of converting to the light olefins in a catalytic reaction or to a clean reaction intermediate compound of the catalytic reaction and converting the oxygenate compound to the clean reaction intermediate compound in a chemical reaction. The chemical reaction produces a gas-phase product comprising the clean reaction intermediate compound, unconverted oxygenate, and a reaction byproduct. The method further includes cooling the gas-phase product to condense the unconverted oxygenate and the reaction byproduct while maintaining the clean reaction intermediate compound in a gas-phase and separating the clean reaction intermediate compound, the unconverted oxygenate, and the reaction byproduct into a first stream comprising the clean reaction intermediate compound and a second stream comprising the unconverted oxygenate and the reaction byproduct. Still further, the method includes heating the first stream and contacting the first stream with a catalyst configured for producing the light olefins from either the clean reaction intermediate compound or the oxygenate compound for a length of time sufficient to heat the catalyst configured for producing the light olefins to a temperature sufficiently high for producing the light olefins from the clean reaction intermediate compound or from the oxygenate compound. The method also includes contacting a third stream comprising the oxygenate compound with the catalyst configured for producing the light olefins.

In accordance with yet another exemplary embodiment, a method includes providing a gas-phase stream comprising the clean reaction intermediate compound and the oxygenate compound at an elevated temperature and cooling the gas-phase stream to condense the unconverted oxygenate while maintaining the clean reaction intermediate compound in a gas-phase. The method further includes separating the clean reaction intermediate compound from the oxygenate compound to produce a clean reaction intermediate compound stream and heating the clean reaction intermediate compound stream using the gas-phase stream. Cooling the gas phase stream and heating the clean reaction intermediate compound stream occur substantially without adding or removing heat outside of the gas-phase stream and the clean reaction intermediate compound stream.

### BRIEF DESCRIPTION OF THE DRAWINGS

The light olefin production systems and methods will hereinafter be described in conjunction with the following drawing figures, wherein like numerals denote like elements, and wherein:

FIG. 1 is a simplified block diagram of a process for the production of olefins in accordance with one exemplary embodiment; and

FIG. 2 is a simplified block diagram of a process for the production of olefins in accordance with another exemplary embodiment.

### DETAILED DESCRIPTION

The following detailed description is merely exemplary in nature and is not intended to limit the olefin production systems and methods or the application and uses of the olefin production systems and methods. Furthermore, there is no intention to be bound by any theory presented in the preceding background or the following detailed description.

The various embodiments contemplated herein relate to methods for the production of light olefins from oxygenates using “clean” reaction intermediate compounds to reduce the cost and complexity of catalytic reactor start-up procedures. The term “clean” as used herein refers generally to a compound that in its gaseous state is suitable as a feed component in a catalytic reactor during start-up procedures. As will be appreciated by those having ordinary skill in the art, the introduction of vaporized oxygenates such as methanol, ethanol, etc., directly into a cold catalytic reactor will result in the cooling and condensing of the vaporized oxygenate on to the catalyst (in addition to the water fraction included in the oxygenate feed). If this occurs, the catalyst will be ineffective at catalyzing the desired reaction because the condensed oxygenate and water will substantially prevent vapor phase oxygenate molecules from reaching the reaction sites on the catalyst. Furthermore, presence of a liquid phase by any such condensation will hinder the fluidization of the catalyst particles. Clean compounds of this process have condensation temperatures (at the reactor pressure employed) sufficiently low that there is no or minimal danger of condensation on the catalyst if fed into a cold catalytic reactor to initiate start-up procedures. In this manner, the clean compound can be directly introduced into a cold reactor to bring the reactor to normal operating temperatures without ruining the catalyst, as well as allowing the catalyst particles to remain in desired fluidized state for the optimal reaction conditions for the oxygenates conversion to olefins.

As previously discussed, conventionally used clean compounds have included natural gas, methane, and nitrogen, among others. However, these compounds are not used in, nor are they reaction intermediates of, catalytic oxygenate-to-olefin reactions, and therefore must be separately provided to start up the reactor before actual olefin production can begin.

The novel methods disclosed herein employ reaction intermediates of the catalytic oxygen-to-olefin reactions that are also clean compounds for use during start-up procedures. Reaction intermediates can be created from the oxygenate feed, and as such do not incur the added expense of using compounds outside of the desired reaction (e.g., nitrogen, natural gas, etc. as currently known in the art). Furthermore, reaction intermediates, as part of the desired oxygen-to-olefin reaction, form olefin products when contacted with the catalyst under appropriate conditions, and therefore can be recycled and eventually converted to product during and after the reactor start-up procedures. This is of great importance and benefit for the conversion of oxygenates to olefins process. In the current state of the art, where nitrogen or natural gas (methane, ethane, or LPG) is used for the fluidization of the catalyst particles and to bring the catalyst bed temperature to the desired 300-350° C. In this mode, once the oxygenate feed is introduced the reaction products exiting the reactor also contains a significant amount of the nitrogen or the natural gas. The light olefins production, reaction intermediate clean compounds, and the nitrogen or natural gas used for the start-up are normally in a gas phase at ambient conditions. If the reactor effluent is recycled, the inert nitrogen or natural gas will continue to stay in the system. Not only that such stream can not be recycled, but it also causes difficulties, if sent to downstream cold separation and product distillation section. Thus, in the current state of the art, it becomes necessary the reactor effluent from the initial start-up phase is sent to a flare until these compounds are purged out of the system, causing a loss of raw material and a potential environmental concern.

While a variety of oxygenates can be selected for use in an oxygenate-to-olefin reaction, the following examples, for simplicity, will refer to the methanol-to-olefin (MTO) reaction, wherein the “clean” reaction intermediate employed during start-up procedures is dimethyl ether (DME). It is expected that a person of ordinary skill in the art, following these examples, will be able to select other oxygenates for the production of olefins using reaction chemistries and reaction schemes that are well known in the art.

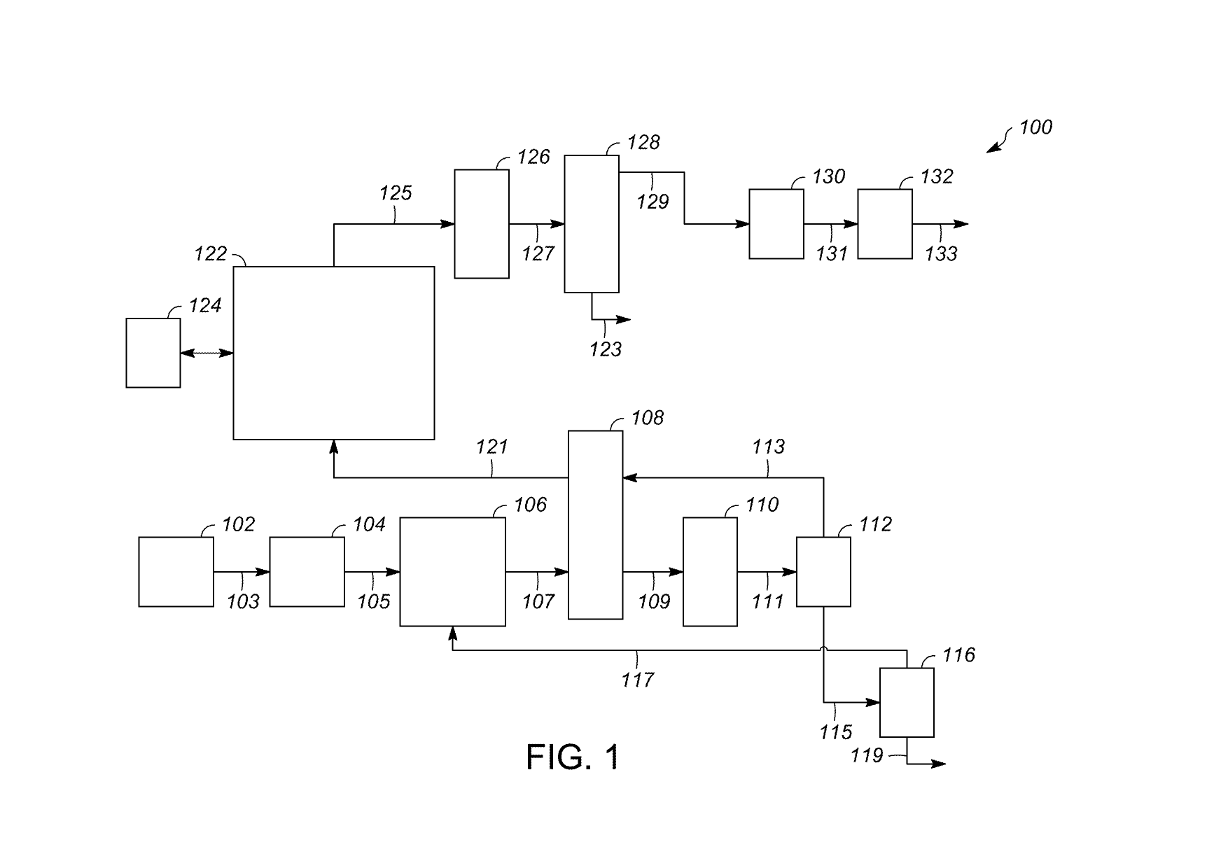

In one embodiment, with reference to FIG. 1, a simplified schematic diagram of a process 100 for the production of olefins is depicted. As shown therein, a methanol feed source 102 is provided as the primary feed material for the production of light olefins. The methanol feed source 102 supplies methanol via stream 103 to vaporizer and pre-heater 104. The vaporizer and preheater 104 vaporizes the methanol and associated water fraction, and brings the vapor to a temperature between about 200-250° C.

The vaporized and heated methanol is then fed via stream 105 to a reactor 106 for the conversion of methanol to the clean reaction intermediate DME. Methanol is converted to DME through the use of an acidic catalyst. While any acidic catalyst could be employed, in an exemplary embodiment the acidic catalyst includes a gamma alumina catalyst. Furthermore, a preferred reactor design includes a fixed bed reactor loaded with a gamma alumina catalyst. The sizing and selection of the reactor for the methanol-to-DME reaction is within the skill of a person having ordinary skill in the art, and depends on production volume requirements, conversion requirements, operating conditions, etc.

The methanol-to-DME reaction proceeds according to the following stoichiometry:

2CH₃OH→1CH₃OCH₃+1H₂O.

The methanol-to-DME reaction is exothermic, resulting in a product stream 107 exiting the reactor at a temperature between about 300 to about 500° C., and preferably between 350 and 400° C. At these reaction conditions, the methanol-to-DME reaction over gamma alumina in a fixed bed reactor proceeds to a conversion of between about 80 to about 90%.

The product stream 107 from the methanol-to-DME reactor 106 proceeds to a heat exchange system, for example a first heat exchanger 108, where the product stream 107 is cooled down to a temperature between about 150 to about 300° C. Heat is exchanged with a feed stream of DME to the olefin production reactor, as will be discussed in greater detail below. A partially cooled product stream 109 then proceeds to a second heat exchanger 110, where the product stream 111 is cooled to a temperature between about 40 to about 50° C. Heat is exchanged in the second heat exchanger with another process stream that requires heating such as preheating oxygenates (methanol) feed to the vaporizer 104, or a separate cooling medium, for example water or air. In such case, the heat exchanger 108 may be split in two, first heat exchanging against a process stream to be heated followed by exchanging against a cooling medium air or water. At 40-50° C., the water byproduct and the un-reacted 10-20% methanol will condense, while the DME will remain in gas phase.

The product stream 111 proceeds to a phase separator 112, where the gas phase DME is diverted to a separate stream 113 from the condensed liquid-phase methanol and water mixture. Phase separation can proceed to substantial completion. For example, phase separation can be greater than 90%, greater than 95%, or greater than 99%. A liquid phase methanol and water stream 115 can be sent to a fractionator 116, where methanol is fractionated from the mixture as an overhead product stream 117 and recycled to the methanol-to-DME reactor 106. Water is removed from the system as a bottoms product 119 of the fractionator 116.

The gas phase DME stream 113 proceeds to the first heat exchanger 108 where it exchanges heat with the product stream 107 from the methanol-to-DME reactor 106. The gas-phase DME stream 113 is brought up to a temperature between about 300 to about 350° C. In this manner, the DME is heated to a temperature suitable for use in starting-up the olefin production reactor using only heat already in the system, and the product stream 107 is cooled so that less external cooling is required in the second heat exchanger 110 to bring the stream 109 to a temperature for condensation of methanol and water. As such, the use of additional energy from an external heat source is avoided to heat the DME to a suitable start-up temperature, and the amount of cooling medium required in the second heat exchanger is minimized. Thus, the first heat exchanger 108 operates substantially without adding or removing heat outside of the product stream 107 and the separated gas-phase DME stream 113.

A heated DME stream 121 from the first heat exchanger 108 then proceeds to the olefin production reactor 122. During start-up operations, the heated DME stream 121 flows through a cold reactor 122 to bring the catalyst contained therein to a temperature high enough to initiate the olefin production reaction, i.e., DME to olefin. DME (or methanol) can be converted to olefins using microporous crystalline zeolite and non-zeolitic catalysts. While any such catalyst can be used, preferred catalysts include silicoaluminophosphate (SAPO) compounds, and more preferably a SAPO-34 catalyst. Such catalysts are preferably employed in a fluidized bed reactor. Under these conditions, the conversion of DME to olefins begins at about 300° C. The sizing and selection of the reactor for the DME to olefins reaction is within the skill of a person having ordinary skill in the art, and depends on production volume requirements, conversion requirements, operating conditions, etc. It is noted however that as the reaction intermediate DME is being converted directly to olefins, a relatively smaller reactor volume (approximately 64% smaller in the present example) is required than for a reactor using methanol to directly produce the same amount of light olefin product.

The DME to light olefin reaction proceeds according to the following stoichiometry:

6CH₃OCH₃→3C₂H₄+2C₃H₆+6H₂O.

The reaction is exothermic, and the fluidized bed reactor 122 can be allowed to rise to a temperature ranging generally between about 400 to about 500° C. It will be appreciated, however, that olefin product ratios can be affected by the operating temperature. For example, if increased ethylene production is desired, then the reactor can be operated at a temperature between about 475 to about 550° C., and for example between about 500 to about 520° C. If increased propylene production is desired, then the reactor can be operated at a temperature between about 350 to about 475° C. and for example between about 400 to about 470° C. By adjusting the reaction temperature, the light olefins produced can have a ratio of ethylene to propylene of in the range from about 0.5 to about 2.0. If a higher ratio of ethylene to propylene is desired, then the reaction temperature is generally desirably higher than if a lower ratio of ethylene to propylene is desired. Once the desired reaction temperature is reached, any additional heat generated by the reaction can be removed by conventional means, including generating steam in a catalyst cooler 124 associated with the reactor 122, for example. Though this is an exothermic reaction, by proper circulation of the catalyst between the catalyst cooler 124 and the reactor 122 the temperature of catalyst bed in the reactor 122 is maintained close to an isothermal condition. That is temperature gradient from the inlet to the outlet of the reactor 122 is maintained to less than 50° C., and preferably is minimal to less than 10° C.

An olefin production reaction product stream 125 from the fluidized bed reactor 122 proceeds to a third heat exchanger 126. As described above the reactor effluent exits the reactor at the temperature of the reaction condition, typically in the range of 400 to 500° C. This stream ultimately needs to be cooled to a temperature sufficient to condense out water of the reaction product. One skilled in the art would know to recover as much heat out of the stream 125 by exchanging with other process stream that may be needed to be heated, or by generating steam in heat exchanger 126. In this mode, most, if not all, most of the exothermic heat of oxygenates to olefins reaction is recovered by steam generated in heat exchanger 126 and the catalyst cooler 124. The stream 127 from the heat exchanger 126 passes to a quench tower 128. In the quench tower 128, the water of the reaction product will condense out in a bottoms stream 123 and be removed from the system. The overhead product stream 129, including primarily light olefins, proceeds via a stream 129 to a caustic scrubber 130 to remove traces of carbon dioxide and other minor acidic compounds. The scrubbed product 131 then proceeds to a compressor 132 for product recovery, resulting in a finished light olefin product stream 133.

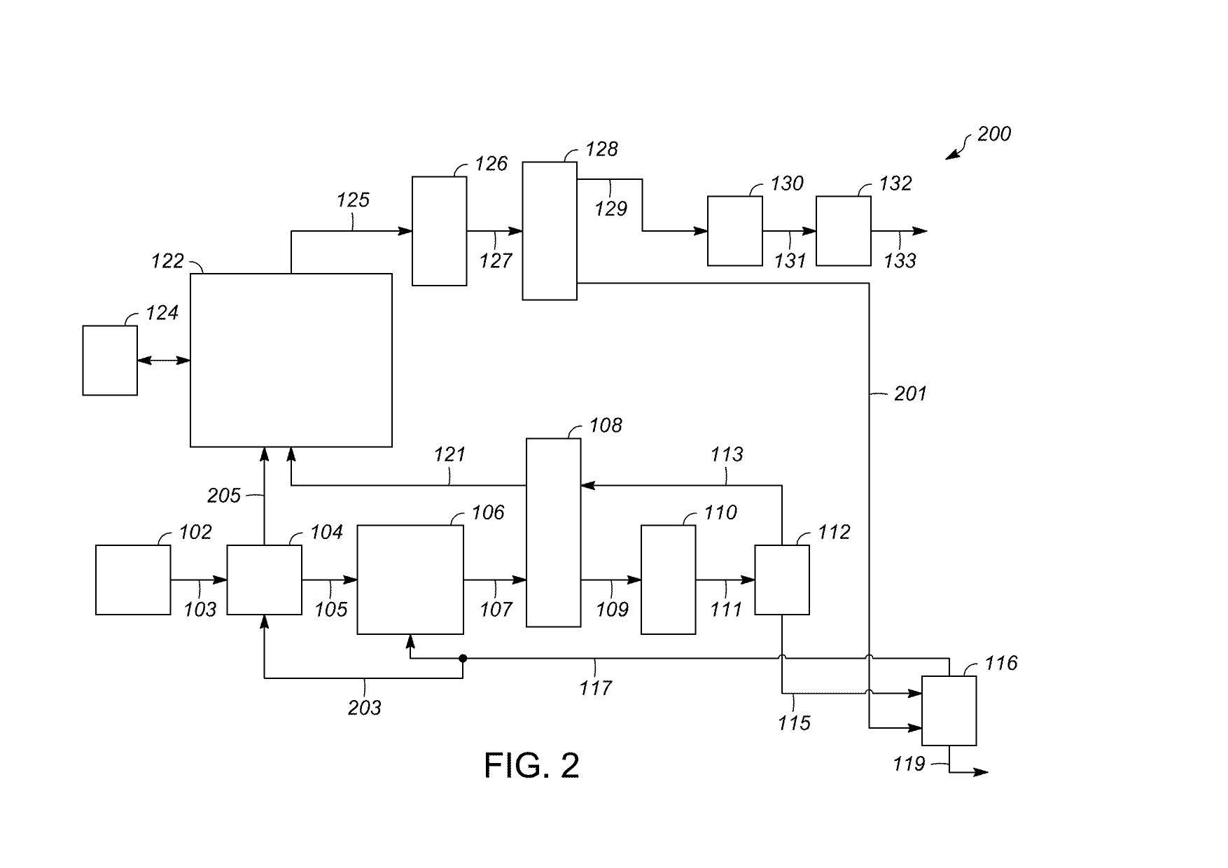

In another embodiment, with reference to FIG. 2, DME is used during start-up procedures as discussed above, but rather than continuing to use DME as the sole feed to the olefin production reactor once the reactor reaches the appropriate temperature, the production of DME is reduced or discontinued and vaporized methanol is introduced directly into the olefin production reactor for the production of olefins directly from methanol (MTO reaction). One skilled in the art of oxygenates to olefins conversion, specifically methanol to olefins would know that increased selectivity to light olefins is achieved at lower partial pressure of hydrocarbon products in the reactor. Thus, having a methanol is beneficial because it adds additional mol of water per mol of methanol feed. Lower partial pressure favors production of ethylene and higher partial pressure favors production of propylene. The MTO reaction proceeds according to the following stoichiometry:

12CH₃OH→6CH₃OCH₃+6H₂O→3C₂H₄+2C₃H₆+12H₂O

Thus, it is desirable to switch from the start-up clean intermediate, DME feed to the vaporized methanol feed.

As shown in FIG. 2, a simplified schematic diagram of a process 200 for the production of olefins is depicted. In this embodiment, the olefin production system design remains largely the same during start-up procedures. Methanol 102 is fed to the vaporizer/preheater 104. The heated and vaporized methanol proceeds to a methanol-to-DME reactor 106, such as a fixed bed reactor with a gamma alumina catalyst. Two heat exchangers 108,110 are employed to cool the methanol-to-DME reaction product stream to a temperature suitable for the condensation of water and un-reacted methanol (the first of which beneficially exchanges heat with the olefin production reactor 122 DME feed stream). Phase separation (112) is then employed to direct a vapor phase DME stream back into the first heat exchanger 108 for heating to a suitable temperature for starting-up the olefin production reactor 122.

In this embodiment, once the olefin production reactor 122 has been brought up to a suitable temperature to initiate the production of olefins, DME production can be reduced or discontinued, and a separate stream 205 of vaporized and heated methanol from the vaporizer/heater 104 can be directed into the olefin production reactor 122 for an MTO reaction In operations where the DME feed to the reactor 122 is reduced and not eliminated completely, the relative ratios of ethylene and propylene produced can be manipulated by varying the methanol/DME feed ratios into the reactor 122.

Once the reaction is complete, the product stream 125, which will also contain some un-reacted methanol, can be sent to the same heat exchanger 126 and quench tower 128 as described previously. In this embodiment, the bottoms product will also contain some methanol with the water. The bottoms stream 201 can be sent to the fractionation column 116, for separation of methanol from the methanol/water mixture. The methanol overhead product can be recycled back into the vaporizer/heater 104 using a separate stream 203, for feeding back into the olefin production reactor 122. The overhead product stream 129 from the quench tower 128, as before, proceeds to a scrubber 130 and compressor 132 to produce the finished light olefin product stream 133.

Accordingly, systems and methods for the production of light olefins have been described. The systems and methods reduce production costs and improve production efficiency by utilizing clean reaction intermediates during reactor start-up procedures. While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. Rather, the foregoing detailed description will provide those skilled in the art with a convenient road map for implementing the described embodiment or embodiments. It should be understood that various changes can be made in the processes without departing from the scope defined by the claims, which includes known equivalents and foreseeable equivalents at the time of this disclosure.

What is claimed is:

1. A method for producing light olefins, comprising:
providing an oxygenate compound capable of converting to the light olefins in a catalytic reaction or to a clean reaction intermediate compound of the catalytic reaction;
converting the oxygenate compound to the clean reaction intermediate compound in a chemical reaction, wherein the chemical reaction produces a gas-phase product comprising the clean reaction intermediate compound, unconverted oxygenate, and a reaction byproduct;
cooling the gas-phase product to condense the unconverted oxygenate and the reaction byproduct while maintaining the clean reaction intermediate compound in a gas phase;
separating the clean reaction intermediate compound, the unconverted oxygenate, and the reaction byproduct into a first stream comprising the clean reaction intermediate compound and a second stream comprising the unconverted oxygenate and the reaction byproduct;
heating the first stream using the gas-phase product; and
contacting the first stream with a catalyst configured for producing the light olefins.

2. The method of claim 1, wherein the clean reaction intermediate compound maintains a gas phase when contacted with the catalyst configured for producing the light olefins during a start-up procedure.

3. The method of claim 2, wherein the oxygenate compound is methanol, the clean reaction intermediate compound is dimethyl ether, and the reaction byproduct is water.

4. The method of claim 1, wherein cooling the gas-phase product comprises cooling the gas-phase product to a temperature below the condensation point of the oxygenate compound and the reaction byproduct and wherein heating the first stream comprises heating the first stream to a temperature sufficient for start-up procedures.

5. The method of claim 4, wherein cooling the gas-phase product comprises cooling the gas-phase product to a temperature to a temperature between about 40 to about 50° C. and wherein heating the first stream comprises heating the first stream to a temperature between about 300 to about 350° C.

6. The method of claim 4, wherein cooling and heating occurs substantially without adding or removing heat outside of the gas-phase product and the first stream.

7. The method of claim 1, wherein converting the oxygenate compound to the clean reaction intermediate compound is performed with the use of an acidic catalyst.

8. The method of claim 7, converting the oxygenate compound to the clean reaction intermediate compound is performed with the use of a gamma alumina catalyst.

9. The method of claim 8, wherein converting the oxygenate compound to the clean reaction intermediate compound occurs with the use of the gamma alumina catalyst.

10. The method of claim 1, further comprising fractionating the second stream to recover the unconverted oxygenate.

11. The method of claim 1, wherein the catalyst configured for producing the light olefins is further configured for converting the clean reaction intermediate compound into the light olefins.

12. The method of claim 11, further comprising converting the clean reaction intermediate compound into the light olefins with the use of the catalyst configured for producing the light olefins.

13. The method of claim 12, further comprising converting the clean reaction intermediate compound into the light olefins with the use of a SAPO catalyst.

14. The method of claim 13, further comprising converting the clean reaction intermediate compound into the light olefins with the use of a SAPO-34 catalyst.

15. The method of claim 1, wherein contacting the first stream with a catalyst configured for producing the light olefins is performed for a length of time sufficient to heat the catalyst configured for producing light olefins to a temperature sufficiently high for producing the light olefins from the clean reaction intermediate compound.

16. The method of claim 15, wherein contacting the first stream with a catalyst configured for producing the light olefins is performed for a length of time sufficient to heat the catalyst configured for producing light olefins to a temperature above about 300° C.

17. A start-up method for use in systems for producing light olefins, comprising:
providing an oxygenate compound capable of converting to the light olefins in a catalytic reaction or to a clean reaction intermediate compound of the catalytic reaction;
converting the oxygenate compound to the clean reaction intermediate compound in a chemical reaction, wherein the chemical reaction produces a gas-phase product comprising the clean reaction intermediate compound, unconverted oxygenate, and a reaction byproduct;
cooling the gas-phase product to condense the unconverted oxygenate and the reaction byproduct while maintaining the clean reaction intermediate compound in a gas-phase;
separating the clean reaction intermediate compound, the unconverted oxygenate, and the reaction byproduct into a first stream comprising the clean reaction intermediate compound and a second stream comprising the unconverted oxygenate and the reaction byproduct;
heating the first stream;
contacting the first stream with a catalyst configured for producing the light olefins from either the clean reaction intermediate compound or the oxygenate compound for a length of time sufficient to heat the catalyst configured for producing the light olefins to a temperature sufficiently high for producing the light olefins from the clean reaction intermediate compound or from the oxygenate compound; and
contacting a third stream comprising the oxygenate compound with the catalyst configured for producing the light olefins while simultaneously discontinuing contacting the first stream with the catalyst configure for producing the light olefins.

18. The method of claim 17, further comprising discontinuing contacting the first stream with the catalyst configured for producing the light olefins prior to contacting the third stream with the catalyst configured for producing the light olefins.

19. The method of claim 17, further comprising vaporizing and heating the oxygenate compound to a temperature between about 200 to about 400° C. prior to contacting the third stream with the catalyst configured for producing the light olefins.

20. A method for producing light olefins using an oxygenate compound capable of converting to light olefins in a catalytic reaction or to a clean reaction intermediate compound of the catalytic reaction, comprising:
providing a gas-phase stream comprising the clean reaction intermediate compound and the oxygenate compound at an elevated temperature;
cooling the gas-phase stream to condense the unconverted oxygenate while maintaining the clean reaction intermediate compound in a gas-phase;
separating the clean reaction intermediate compound from the oxygenate compound to produce a clean reaction intermediate compound stream; and
heating the clean reaction intermediate compound stream using the gas-phase stream, wherein cooling the gas phase stream and heating the clean reaction intermediate compound stream occur substantially without adding or removing heat outside of the gas-phase stream and the clean reaction intermediate compound stream.
